(19) United States
(12) Patent Application Publication
(10) Pub. No.: US 2007/0087002 A1
(43) Pub. Date: Apr. 19, 2007

(54) Anticholesterol immunoglobulin to treat lipid raft diseases

(76) Inventor: Shawn J. Green, Davis, CA (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/581,773

(22) Filed: Oct. 16, 2006

Related U.S. Application Data
(60) Provisional application No. 60/727,048, filed on Oct. 14, 2005.

(51) Int. Cl. A61K 39/395

(52) U.S. Cl. 424/145.1

(57) ABSTRACT

Immunoreactive compositions and methods for immunizing animal, including, humans, cows, and fowl, against cholesterol and cholesterol derivatives, including cholesterol oxides, and their use in methods for reducing and preventing lipid raft-based diseases, including, but not limited to HIV-1, SARS, prion formation in Creutzfeldt-Jakob disease, and neutralizing oxidized modified lipoproteins, specifically, oxidized-LDL, oxidized-VLDL/IDL, and oxidized-chylomicrons, which contribute to the formation of fatty streaks and atherosclerotic plaques, are described.

### CROSS-REFERENCE TO RELATED APPLICATIONS

This application claims the benefit of U.S. Provisional Patent Application No. 60/727,048, filed Oct. 14, 2005, which is incorporated herein by reference in its entirety.

### FIELD OF THE INVENTION

The present application relates to compositions and methods for reducing, preventing and treating lipid raft-based diseases, which contribute to the formation of fatty streaks and atherosclerotic plaques.

### BACKGROUND OF THE INVENTION

By targeting uniquely displayed self-antigens, specifically cholesterol-rich domains, lipid raft diseases can be reduced, prevented or treated with anti-lipid antibodies, such as the antibodies to cholesterol, cholesterol oxides, and cholesterol sterol-like molecules.

Lipid raft diseases are characterized by membrane surface domains containing phospholipids and/or sphingolipids and cholesterol, referred to as membrane or lipid rafts, which have unique physiochemical properties. For example, domains are typically resistant to detergents solubilization of 4° C. and destabilized by cholesterol and/or sphingolipid depleting agents. Lipids rafts have been morphologically characterized as small membrane patches that are ten to less than a 100 nanometer in diameter. These rafts, in context of this application, are portals of entry for various pathogens and toxins, such as HIV-1, and serve as preferential sites of formation of prions for Creutzfeldt-Jakob (Mad Cow Disease), and beta-amyloid peptide associated with Alzheimer's disease. Lipid rafts also act as molecular sorting machines capable of coordinating transduction signals with selected areas such as signalosomes.

Lipid rafts are important for several functions that include transporting materials into, out of, and throughout the cell. They are important not only for HIV, but other viruses such as influenza and measles, and chronic disease, including the uptake of oxidized or modified LDL by macrophages and endothelial cells at sites within the vascular system that leads to athrosclerolotic plaques.

Disease for which lipid rafts and raft membrane components are targets include, but are not limited to, Alzheimer's, muscular dystrophy, asthma, neoplasia, atherosclerosis, and various infections, specifically, influenza, SARS, HIV-1, measles, RSV, filoviridae, papillomaviridae, Epstein-Barr, M tuberculosis, V. cholerae, C. difficile, C. tetani, Salmonella, Shiella, malaria, sleeping sickness, toxoplasma, and prion formation, including Creutzfeldt-Jakob.

Numerous reports over the past 80 years have shown that anti-cholesterol antibodies can be induced in animals and naturally-occurring antibodies exist in both animals and humans without any evidence of disease or autoimmunity.

It appears that the prerequisite for induction and binding of anti-cholesterol antibodies resides in membrane cholesterol density. Although immuno-based strategies have been geared towards cholesterol reduction, no teachings suggests that anti-cholesterol antibodies may be used against non-cardiovascular diseases, such as HIV-1, SARS, and prion formation.

In healthy cells, cholesterol is cryptic; it is buried between phospholipids that make up the lipid membranes, and cholesterol is seldom exposed, thereby remaining immuno-silent. However, by exposing cholesterol in high density liposome in the presence of an adjuvant, cholesterol was rendered immunogenic. The present application recognizes that immunoreactive serum and a monoclonal antibody to cholesterol were found to react with membranes containing cholesterol-rich domains, thereby explaining why healthy mammalian cells, HDL, and 43% cholesterol-deficient liposomes are protected from these antibodies, while LDL, VLDL, and 71% cholesterol-rich liposomes (and possibly, Mycoplasma) are highly reactive to anti-cholesterol antibodies resulting in membrane destabilization.

Thus far, no teaching exists suggesting that anti-cholesterol antibodies or antibodies against sterol have any effect on or treatment benefit in lipid raft diseases, such as HIV and reducing oxidized lipoproteins.

For illustrative purposes, the role of lipid rafts in HIV budding demonstrates the importance of this cholesterol-rich site in lipid raft diseases. Using various compounds to change cholesterol levels, such as beta-cyclodextrin, cells become resistant to HIV infection or released non-infectious HIV particles.

Although data is limited on the lipid arrangement of cholesterol and phospholipids within the HIV envelope, it is reasonable to assume that switching of cholesterol from the mammalian cell membranes to virus membranes results in exposed cholesterol-rich rafts, thereby, eliciting an immune response. Although not presently recognized in publication and patents as a potential target, lipid rafts are unique site for immuno-based therapeutic strategies.

Recent reports have shown that serum levels of anti-cholesterol antibodies are elevated in HIV patients, and this serum diminishes the binding of HIV virions to cholesterol-coated plates, however, such studies and observations provide no teaching as to the utility of anti-cholesterol.

For illustrative purposes, the role of high density cholesterol domains, reminiscent of lipid raft, are found on low density lipoproteins which are involved in the uptake of lipoproteins by macrophages and endothelial cells at athrosclerotic sites in blood vessels. Previous studies demonstrated that anti-cholesterol antibodies bind preferentially to LDL, VLDL, and IDL. More recent studies have shown that hyperimmune and some sources of naive cow milk and colostrum or whey contains immunoglobulin protein containing specific binding activity to cholesterol, LDL, IDL, VLDL, and chylomicrons as described in U.S. patent application Ser. No. 11/165,601, which is incorporated by reference herein.

Thus far, no scientific research has recognized the beneficial value of antibodies specific for cholesterol (found in immune serum and ascites, naïve or hyperimmune milk and colostrum) beyond cholesterol lowering activity. The object of all prior scientific studies has focused on the cholesterol-lowering properties of anticholesterol immunoglobulins to control hypercholesterolemia and atherosclerosis.

Atherosclerosis and its complications, such as myocardial infarction, stroke and peripheral vascular disease, are a major cause of death in the United States and Western Europe. The first type of lesion seen, referred to by those skilled in the art as fatty streaks, are grossly visible, raised, yellow areas that consist of subendothelial foam cells (lipid-filled cells derived from macrophages and smooth muscle cells) and some leukocytes. The second type of atherosclerotic lesion, which causes narrowing of the vessel and predisposes the vessel to thrombosis and calcification, is the fibro-fatty plaque. A typical plaque consists of a fibrous cap composed of smooth muscle cells, a few leukocytes and dense extracellular material. A cellular area beneath the cap generally consists of macrophages, foam cells, smooth muscle cells, leukocytes, cellular debris, extracellular lipid, cholesterol crystals and calcium deposits. The cholesterol that accumulates in both of these types of atherosclerotic lesions originates primarily in plasma lipoproteins, predominantly low LDL.

Elevated levels of plasma LDL are associated with accelerated atherosclerosis. There is growing evidence supporting the hypothesis that oxidative modification of preferentially small, dense LDL renders it more atherogenic. Uptake of native LDL does not appear to be responsible for LDL accumulation in the lesion. However, when native LDL is modified, by oxidation for example, it is recognized and taken up by the scavenger receptor, a specific receptor distinct from the LDL receptor. The scavenger receptor recognizes chemically modified LDL including acetylated LDL, malondialdehyde-conjugated LDL, as well as LDL modified by cultured endothelial cells, monocytes and smooth muscle cells. This receptor is found on monocyte/macrophages, endothelial cells and smooth muscle cells. Thus modified LDL is a key element in plaque formation and/or progression. Markers or methods to detect modified LDL would provide valuable diagnostic and/or prognostic tools.

No teaching thus far suggests that anti-cholesterol antibodies or antibodies against sterol have any effect or treatment, i.e., clearance of oxidized or modified LDL or shift athrogenic small dense LDLNVLDL to less athrogenic large forms of the lipoprotein.

### SUMMARY OF THE INVENTION

A composition containing an immunoglobulin protein is provided for use in passive immune therapy either administered intravenously, topically, or orally or as an food supplement and ingredient that is convenient and economical to administer, and that is derived from naive or hyperimmune milk, whey, or serum and certified via an immunoassay for detection of specific antibodies to cholesterol to treat lipid-based disease, including but not limited to, HIV-1, SARS, prion formation, and atherosclerosis.

An antibody specific for cholesterol, either polyclonal or monoclonal, and either mouse or humanized antibody, is described herein and is useful for the reduction, prevention or treatment of lipid raft diseases.

Also provided is a diagnostic or marker of lipid raft diseases.

Use of anticholesterol immunoglobulins as vaccines for prophylactic or therapeutic strategy to prevent or treat lipid raft diseases is also described.

### DETAILED DESCRIPTION OF THE PREFERRED EMBODIMENT

The elicitation of self or autoantibodies may be necessary and warranted in designing a durable vaccine or passive transfer strategy against lipid raft diseases, including HIV-1, SARS, prion formation, Alzheimer, Parkinson, atherosclerosis, among other disease that exploit cholesterol rich membrane domains.

Antibodies against components in the host membranes are often conserved, i.e., in virus envelopes and can serve as potentially critical target of viral entry in the host and may be an important consideration in the design of a successful anti-HIV strategy or vaccine against lipid raft dependent disease, including, but not limited to specific diseases associated with lipid rafts. Furthermore, self-antibodies, especially anti-lipid antibodies against cholesterol and cholesterol oxides, are useful in the reduction, prevention, treatment and diagnostic of these diseases.

The anti-cholesterol immunoglobulin are administered to lipid raft disease patients topically, orally, or intravenously, or are administered as a vaccination and boost or are provided as a food ingredient. The patients to whom the immunoglobulins are administered include, but are not limited to, AIDS, SARS, and cardiovascular patients.

Others skilled in the art have not recognize the need to use an immunoassay or ELISA (enzyme-linked immunosorbent assay) to detect, measure, and certify the bio-active presence of anticholesterol antibodies in fluids beyond the role of such immunoassays in cholesterol management in cardiovascular disease.

An object of the composition and method described herein is to provide an immunoglobulin protein as a biologic in passive immune therapy either administered intravenously, topically, or orally or as an food supplement which is convenient and economical to administer, and which is derived from naive or hyperimmune milk and certified via an immunoassay for detection of specific antibodies to cholesterol to treat lipid-based disease, including, but not limited to, HIV-1, SARS, and prion formation.

A further object is to provide a monoclonal antibody to cholesterol either as a mouse or humanized antibody.

Another object is to provide a vaccine to induce anticholesterol immunoglobulins as a prophylactic or therapeutic strategy to reduce, prevent or treat lipid raft diseases.

Albeit substantial evidence has been collected in animal studies that support an immunomodulating effect of orally-administered plasma proteins, others have never attempted to use anti-cholesterol antibodies from dairy or milk-derived sources, such as colostrum and whey, to control lipid raft based diseases.

Hybridoma cell lines are also provided that produce anti-cholesterol antibodies, hyperimmune serum, and sources of naturally-occurring antibodies to cholesterols found in whey and dairy sources that react with the atheroma-associated liposomes and with oxidized forms of low density lipoproteins four times greater than to native LDL, VLDL/IDL.

Furthermore, anticholesterol antibodies reacted with cholesterol-rich, low density lipoprotein by increasing the particles size by a minimum of two to four times its native size through the aggregation and subsequent fusion of membrane, thereby resulting in larger, less stable lipoprotein.

There is no teaching thus far suggesting that anti-cholesterol antibodies or antibodies against sterol have any beneficial effect on protecting the host against oxidized, small athrogenic LDL/VLDL, i.e., neutralization of oxidized or modified LDL and shifting small dense LDL/VLDL athrogenic forms to larger LDL/VLDL forms.

Naturally occurring, minimally modified LDL molecule are present in human atherosclerotic plaque as well as in the plasma and serum of a high percentage of patients with advanced coronary artery disease.

The following examples illustrate but do not limit the compositions and methods described herein. Thus, the examples are presented with the understanding that modifications may be made and still be within the spirit of the invention.

### EXAMPLE 1

Anti-Cholesterol Antibodies Disrupt HIV-1 Envelope

Increased concentrations of the anti-cholesterol antibody described herein was able to induce aggregation of HIV-1 virion membranes containing cholesterol-rich microdomains, followed by hemifusion resulting in the disruption of the envelope, which can be further weakened by neutralizing antibodies to HIV. The presence of excess antibodies was also found to prevent entry into target host cells, as well as, the budding of virion particles.

Anti-cholesterol IgM antibodies were found to be a membrane disrupter as cholesterol is incorporated and rearranged into the HIV envelope. Anti-cholesterol antibodies interfere with HIV-gag-raft, inhibit virus cell fusion and assembly, and prevents particle production and virus infectivity.

### EXAMPLE 2

Anti-Cholesterol Antibodies Prevent Uptake of Oxidized LDL and Shifts\Small, Dense LDL to Larger, Less Athrogenic Forms

Oxidative modification of LDL generates a new epitope on the LDL, which is specifically recognized by macrophage receptors. The antibodies described herein recognize a neoepitope generated upon modification of LDL as demonstrated by Western blotting and enzyme linked immunoassay (ELISA). These antibodies do not react with native LDL but do react with malondialdehyde-conjugated LDL and acetylated LDL.

Having described the invention with reference to particular compositions, methods for detection, and sources of anticholesterol activity, i.e., antibodies to cholesterol, and proposals of effectiveness and the like, it will be apparent to those skilled in the art that it is not intended that the compositions and methods described herein be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence that are effective to meet the objectives there intended, unless the context specifically indicates to the contrary.

What is claimed is:

1. A method for reducing or treating lipid raft disease comprising administering to an animal having lipid raft disease an anticholesterol immunoglobulin.

2. The method of claim 1, wherein the animal is a bovine, avian or human.

3. The method of claim 1, wherein the immunoglobulin, is detected by an ELISA or other immunoassay comprised of, but not limited to, cholesterol, cholesterol sterol-like molecules, and cholesterol oxide derivatives, or oxidized lipoproteins consisting of oxidized-LDL, -VLDL, and -chylomicrons.

4. The method of claim 1, wherein the immunoglobulin is used the treatment or prevent of lipid raft diseases, including: Alzheimer, muscular dystrophy, asthma, neoplasia, atherosclerosis, and various infections, specifically, influenza, SARS, HIV-1, measles, RSV, filoviridae, papillomaviridae, Epstein-Barr, M. tuberculosis, V. cholerae, C. difficile, C. tetani, Salmonella, Shiella, malaria, sleeping sickness, toxoplasma, and prion formation in Creutzfeldt-Jakob.

5. The method of claim 1, wherein the immunoglobulin is formulated into a package for internal consumption including an acceptable carrier, including fluid milk/colostrum, powdered milk/colostrum, and any beverage and food.

6. The method of claim 1, wherein the immunoglobulin is formulated for passive transfer immunotherapy or passive immunization, as such, package for internal administration including an acceptable pharmaceutical carrier for intravenous and oral administration.

7. The method of claim 1, wherein the immunoglobulin is formulated for topical, local immunotherapy, as such, applied topically for skin and mucosal surfaces and made into a douche for vaginal administration.

8. The method of claim 1, wherein the immunoglobulin is derived from serum, milk, or colostrum from naïve or hyperimmunized animals, including, but not limited to cows and humans.

9. The method of claim 1, wherein the immunoglobulin of the hyperimmunized animals is enriched by the administration of an immunostimulating agents, including, but not limited to, monophosphoryl lipid A liposomes containing cholesterol or other sterol-like molecule.

10. The method of claim 1, wherein the immunoglobulin is obtained from hyperimmune serum, hybridomas and monoclonal antibodies, including ATCC HB 8995 (monoclonal to cholesterol), and ATCC 23064 and 15539 (Mycoplasma) as is or humanized for passive immunotherapy or diagnostic purposes.

11. The method of claim 1 wherein the immunoglobulin is administered with antiphospholipids, antimicrobials, statins, plant sterols, plant stanols, chemotherapy, antibiotics, subunit vaccines, multivalent vaccines, and other treatment strategies comprising drugs and biologics designed or used to treat lipid raft-based diseases.

12. A method for detecting lipid raft disease comprising combining a biological sample from a patient with an anticholesterol immunoglobulin, wherein the immunoglobulin recognizes oxidized forms of lipoproteins, including oxidized LDL or athroma-derived cholesterol liposomes.

13. A method for reducing or treating lipid raft disease comprising administering to an individual having lipid raft disease an effective amount of an anticholesterol immunoglobulin to immunize the mammal or fowl against lipid raft diseases as measured by a greater than 10-fold increase in relative binding activity for anticholesterol antibodies with a correlative increase in the protective state as shown by a decrease in clinical outcomes of a specific lipid raft disease, including, a decrease in viral burden, athroma thickening, or other associated clinical marker indicative of the progression of disease.

14. The method of claim 12, wherein the lipid raft disease is Alzheimer's Disease, Parkinson's Disease, muscular dystrophy, asthma, neoplasia, atherosclerosis, or an infection, such as influenza, HIV-1, measles, RSV, filoviridae, papillomaviridae, Epstein-Barr virus, M tuberculosis, V. cholerae, C. difficile, C. tetani, Salmonella, Shiella, malaria, sleeping sickness, toxoplasma, or prion formation including Creutzfeldt-Jakob Disease.

15. The method of claim 13, wherein the immunoglobulin is administered for passive immunotherapy for lipid raft disease.
